(19) United States
(12) Patent Application Publication
(10) Pub. No.: US 2008/0064754 A1
(43) Pub. Date: Mar. 13, 2008

(54) Amino ion releasing hydrogen bonded molecule

(76) Inventor: Steven R. Schutt, Prescott, AZ (US)

Correspondence Address: John R. Ross, PO Box 2138, Del Mar, CA, 92014, US

(21) Appl. No.: 11/520,208

(22) Filed: Sep. 12, 2006

(51) Int. Cl. A61K 31/198; C07C 279/12

(52) U.S. Cl. 514/565; 562/439

(57) ABSTRACT

A free amino ion releasing molecule useful for a wide variety of medical and cosmetic applications. The chemical name of the new molecule in acetate form name is 2-amino 5-triaminoguanidovalericbenzoicdiacetate, sometime referred to herein as Tri-Amino. Its chemical formula is C₁₅H₂₈N₆O₆. Its chemical structure is described in the drawings. Two methods of preparing the new amino ion releasing molecule are described.

The present invention relates to amino acids and products that release free amino ions.

### BACKGROUND OF THE INVENTION

### Amino Acids

An amino acid is any organic compound that contains both an amino (—NH₂) and a carboxyl (—COOH) group. There are 20 alpha amino acids from which proteins are synthesized. The names, symbols and structural formulas of these amino acids are shown in FIGS. 1A and 1B.

### Arginine

Arginine is an amino acid. It is produced in the human body by the digestion of proteins. It is also referred to as 2-amino-5-guanidinovaleric acid. Its structure is described in FIG. 1. It is commercially available in pharmaceutical grades from suppliers such as Sigma Aldrich Corporation with offices in St Louis, Mo.

### PABA

p-aminobenzoic acid (PABA) is an organic compound with the molecular formula C₇H₇NO₂. Its structural formula is shown in FIG. 2. It is a white crystalline substance slightly soluble in water. It consists as shown in FIG. 2 of a benzene ring substituted with an amino group and a carboxylic acid. PABA is commercially available in pharmaceutical grades from suppliers such as Sigma Aldrich.

### Reaction Product of Arginine and PABA

In 1995 Applicant filed a patent application describing a reaction product called arginine aminobenzoate made by combining the amino acid arginine with p-aminobenzoic acid. U.S. Pat. Nos. 5,734,080, 6,365,167 and 6,585,988 were issued based on disclosures in the 1995 applications, the last two of which were based on continuation in part applications. Arginine aminobenzoate was prepared by dissolving 10 grams of p-aminobenzoic acid in 50 ml isopropanol at 60 degrees C. with agitation. Then slurry of 10 g of 1-arginine in 50 ml of isopropanol at 60 degrees C. was added to the solution. The reaction product was filtered to yield 14 g of the arginine aminobenzoate. A variety of compositions combining the reaction product with other active and inactive ingredients are described in the three patents (all of which are incorporated herein by reference) and a wide variety of uses of the product was described. These included treatment of wounds, sunburn, non-specific vaginitis, dermatitis and pyoderma in humans and animals and a list of other ailments in animals including saddle sores in horses and udder edema in cattle, goats and sheep.

### Glycine

Glycine is the simplest of the 20 amino acids shown in FIG. 1A. Its chemical name is aminoacetic acid. It is an inhibitor of neurotransmitters in the central nervous system. It is available as a dietary supplement and a gastric antacid and is utilized in bladder irrigation. Studies have indicated that glycine itself may be useful in the treatment of conditions as varied as schizophrenia and cancer. Glycine is commercially available in pharmaceutical grades from suppliers such as Sigma Aldrich.

What is needed is a better product for providing free amino ions at locations in or on the bodies of humans or animals to promote healing or other medical or cosmetic objectives.

### SUMMARY OF THE INVENTION

The present invention provides a free amino ion releasing molecule useful for a wide variety of medical and cosmetic applications. The chemical name of the new molecule in acetate form name is 2-amino 5-triaminoguanidovalericbenzoicdiacetate, sometime referred to herein as Tri-Amino. Its chemical formula is C₁₅H₂₈N₆O₆. Its chemical structure is described in the drawings. Two methods of preparing the new amino ion releasing molecule are described.

The first method includes the steps of mixing arginine with water then adding glycine. Both steps were carried out under specific conditions to produce an intermediate product, 2-amino 5-diaminoguanidovalericdiacetate. The 2-amino 5-diaminoguanidovalericdiacetate is then mixed with p-aminobenzoic acid (PABA) under specific conditions to yield the final product of the present invention, namely 2-amino 5-triaminoguanidovalericbenzoicdiacetate.

The second method includes the steps of mixing glycine with water under specific conditions and then combining the resulting product with p-aminobenzoic acid (PABA) to produce an intermediate product, diaminobenzoic acetate. The diaminobenzoic acetate is then combined with arginine under specific conditions to yield the final product of the present invention, namely 2-amino 5-triaminoguanidovalericbenzoicdiacetate.

The disclosure includes descriptions of a variety of compositions utilizing the final product 2-amino 5-triaminoguanidovalericbenzoicdiacetate. Also disclosed are preliminary results of experiments utilizing the final product in a limited number of tests.

Both of the intermediate products, 2-amino 5-diaminoguanidovalericdiacetate and diaminobenzoic acetate are to the best of the Applicant's knowledge new and useful products and are considered a part of the present invention.

### BRIEF DESCRIPTION OF THE DRAWINGS

FIGS. 1A and 1B are charts showing details of 20 amino acids.

FIG. 2 shows the chemical structure of PABA.

FIG. 3 is a drawing showing the chemical structure of an intermediate product, C₈H₁₉N₅O₄

FIG. 4 is a drawing showing the chemical structure of an intermediate product, C₉H₈N₂O₂

FIG. 5 is a drawing showing the chemical structure of a final product, C₁₅H₂₈N₆O₆

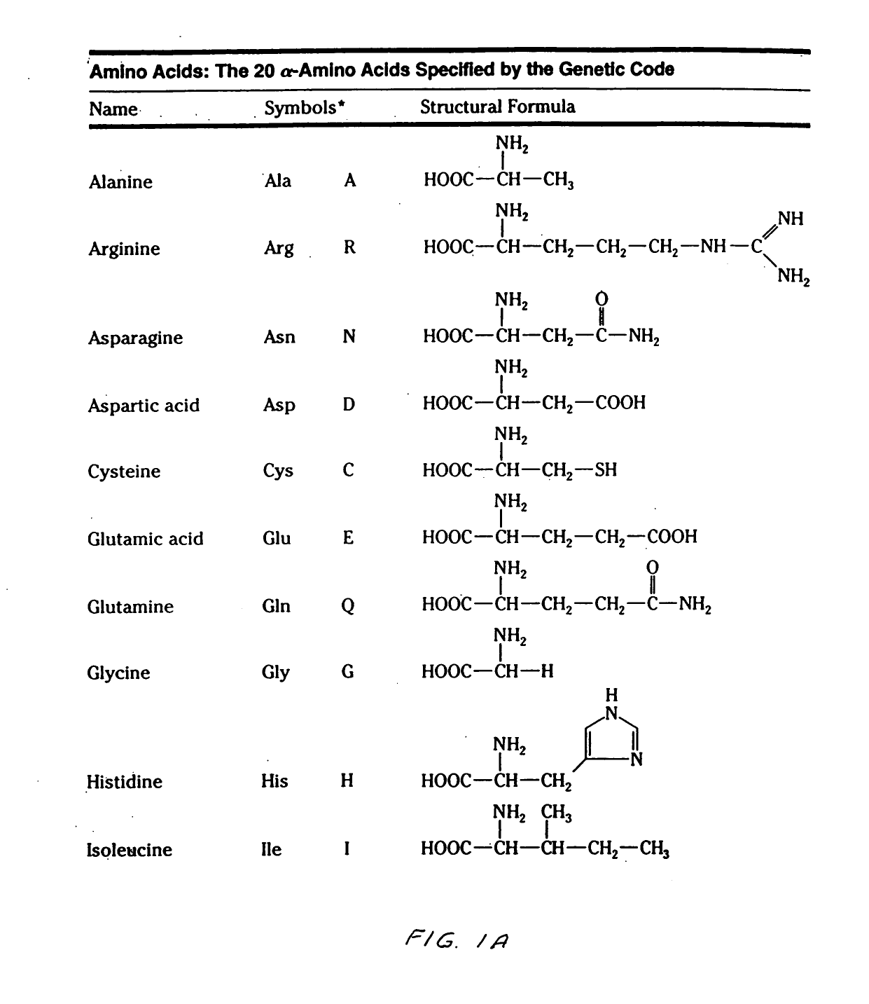

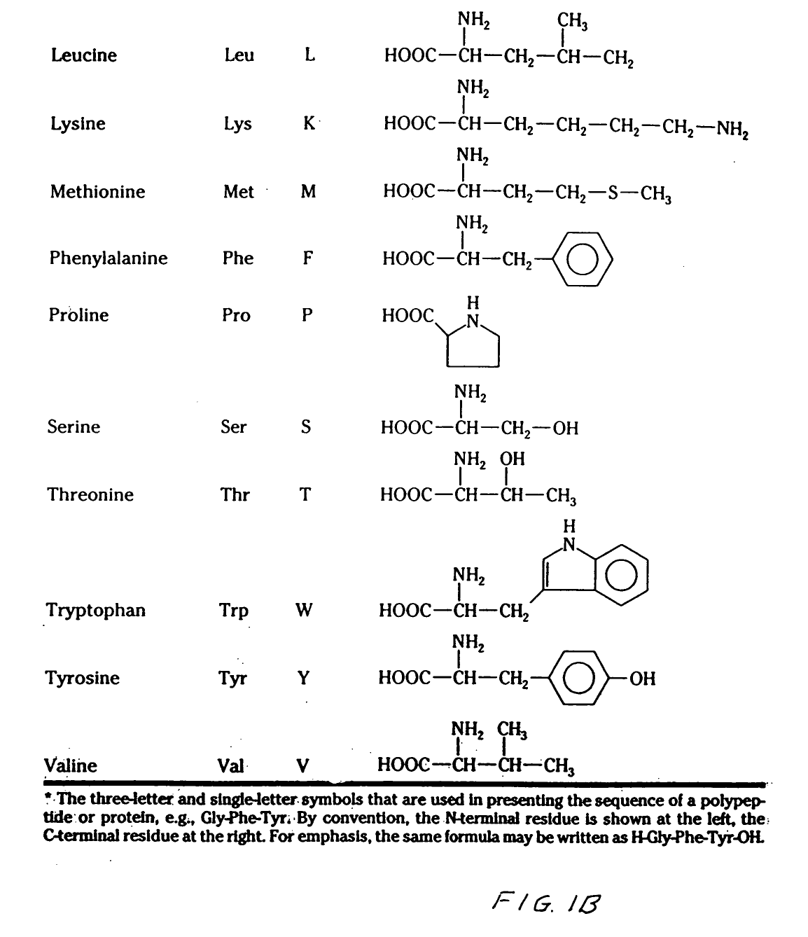

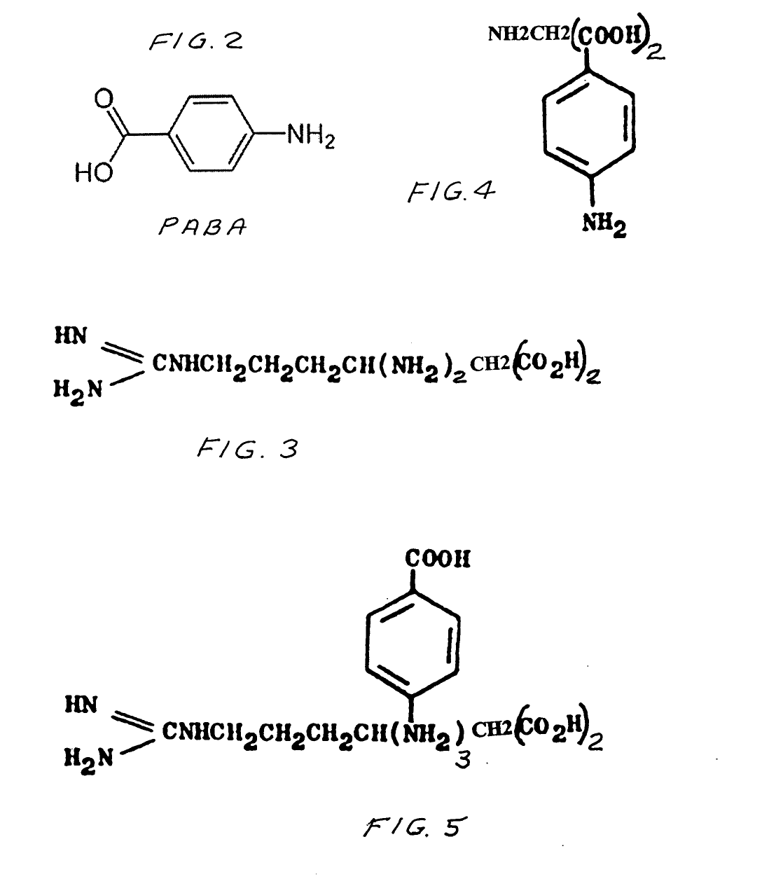

### DETAILED DESCRIPTIONS OF PREFERRED EMBODIMENTS

### Theory

Applicant believes that unstable nucleic acids within mutant cell nuclei cause the mutation of those cells. He also believes that when dysfunctional cells, whose nuclei contain unstable nucleic acids, react with free amino ions in an aqua's solution the resulting biosynthesis causes alignment and stabilization of those cells. Applicant also believes that the process of free amino ions bonding to unstable nucleic acids in the nuclei within the mutant cells promotes this alignment and stabilization. He also believes that it is then possible to cause the mutant cells to return to their normal function as programmed by their original DNA coding. In addition he believes that biosynthesis occurs when free amino ions contact and react with unstable nucleic acids causing them to align and stabilize. A prime example is amniotic fluid. Applicant believes the amniotic fluid keeps the proteins within fetal cell clusters stable as the result of an overwhelming quantity of free amino ions causing constant biosynthesis.

### First Process for Preparing 2-amino 5-triaminoguanidovalericbenzoicdiacetate

### C₁₅H₂₈N₆O₆

### Intermediate Reaction Product,

### 2-amino 5-diaminoguanidovalericdiacetate

### C₈H₁₉N₅O₄

2-amino 5-diaminoguanidovalericdiacetate is an intermediate reaction product and is a unique molecule useful in releasing free amino ions, which because of weak internal hydrogen bonding, then allows for the release of the amino ions. While in aqua's solution these ions provide numerous and various positive physiological effects in both humans and animals such as stabilizing unstable nucleic acids within dysfunctional cells by process of biosynthesis. This reaction product may also be used to boost and accelerate topical microcirculation and is also useful in the manufacture of other reaction products that allows for release of a greater quantity of free amino ions when in aqua's solution to further achieve more effective and positive physiological effects such as the above stated stabilization of unstable nucleic acids within dysfunctional cells by means of biosynthesis.

To obtain 2-amino 5-diaminoguanidovalericdiacetate, those skilled in the art will dissolve at ultra high shear and at 80 degrees Centigrade, 227.0 Grams of 2-amino 5-aminoguanido valaric acid in 1 liter of de-ionized water (USP). The ultra high shear mixing should take at least four minutes. Applicant uses a Ross homogenizer; however a Fischer PowerGen Model 1800D homogenizer would work satisfactorily. The mixing speed should be at least 4,000 rpm. The resulting solution should have PH of about 11.5. Reduce the mixing speed to 300 rpm and add 183.0 Grams of aminoacetic acid. Gradually increase mixing speed to 4000 rpm and mix at this speed for at least four minutes. PH should be about 9.2 but will rise to about 9.3 as solution cools.

A drawing of the chemical structure of this molecule is shown in FIG. 3. This solution of 2-amino 5-diaminoguanidovalericdiacetate may then be used to achieve the final reaction product, namely 2-amino 5-triaminoguanidovalericbenzoicdiacetate.

### Final Reaction Product

### 2-amino 5-triaminoguanidovalericbenzoicdiacetate

To obtain 2-amino 5-triaminoguanidovalericbenzoicdiacetate using the first intermediate reaction product add to the 2-amino 5-diaminoguanidovalericdiacetate solution at 80 degrees Centigrade, 102.0 grams of p-aminobenzoic acid (PABA). Gradually increase mixing speed to 3000 rpm and mix at this speed for at least three minutes. PH should be about 8.3 but will rise to about 9.03 as solution cools to room temperature. Return temperature to 80 degrees C. and add 25 grams of p-aminobenzoic acid again, mixing at 3000 rpm for at least three minutes. The resulting PH should be about 8.2 rising to about 8.5 as the mixture cools to room temperature. This is the satisfactory end point product, i.e. 2-amino 5-triaminoguanidovalericbenzoicdiacetate. A drawing of this hydrogen bonded molecule is shown in FIG. 5.

### Second Process for Preparing 2-amino 5-triaminoguanidovalericbenzoicdiacetate

### Intermediate Reaction Product,

### diaminobenzoicdiacetate

### C₉H₈N₂O₂

Diaminobenzoicdiacetate is an intermediate reaction product and unique motity useful in releasing free amino ions. Weak internal hydrogen bonding allows for the release of those free amino ions that while in aqua's solution provide numerous and various positive physiological effects in both humans and animals such as stabilizing unstable nucleic acids within dysfunctional cells by process of biosynthesis.

This reaction product is useful for accelerating topical microcirculation. It is also useful in the manufacture of other reaction products that release a greater quantity of free amino ions when in solution to further achieve more effective and positive physiological effects such as the above stated stabilization of unstable nucleic acids within dysfunctional cells by means of biosynthesis.

To obtain diaminobenzoicdiacetate, dissolve, at ultra high shear and at 80 degrees Centigrade, 183.0 grams of aminoacetic acid (glycine) in 1 liter of de-ionized water (USP). The high shear mixing should take at least four minutes. The mixing speed should be at least 4,000 rpm. The resulting solution should have PH of about 10.3. Reduce the mixing speed to 300 rpm and add 102.0 grams of p-aninobenzoic acid. Gradually increase mixing speed to 4000 rpm and mix at this speed for at least four minutes. PH should be about 9.1 but will rise to about 9.2 as solution cools.

A drawing showing the chemical structure of this molecule is shown in FIG. 4. This solution of diaminobenzoicdiacetate may then be used to achieve the final reaction product, namely 2-amino 5-triaminoguanidovalericbenzoicdiacetate.

### Final Reaction Product

### 2-amino 5-triaminoguanidovalericbenzoicdiacetate

To obtain the final product, 2-amino5-triaminoguanidovalericbenzoicdiacetate using the diaminobenzoicdiacetate, add to the diaminobenzoicdiacetate solution at 80 degrees Centigrade 227.0 Grams of 2-amino5-aminoguanidovaleric acid (arginine). Gradually increase mixing speed to 3000 rpm and mix at this speed for at least three minutes. PH will be 9.0 but will rise to 9.03 as solution cools to room temperature. Return temperature to 80 degrees C. and add 25 Grams of 4-aminobenzoic acid again, mixing at 3000 rpm for at least three minutes. The resulting PH should be 8.2 rising to 8.5 as the mixture cools to room temperature. This is a satisfactory end point resulting in the final product, i.e. 2-amino 5-triaminoguanidovalericbenzoicdiacetate.

### The Final Reaction Product

### 2-amino 5-triaminoguanidovalericbenzoicdiacetate

Tri-Amino (i.e. 2-amino 5-triaminoguanidovalericbenzoicdiacetate) is a reaction product and unique hydrogen bonded molecule which because of weak internal hydrogen bonding allows for the release of free amino ions. While in aqua's solution, these ions provide numerous and various positive physiological effects in both humans and animals such as stabilizing unstable nucleic acids within dysfunctional cells from the resultant biosynthesis.

### The Desiccate

To desiccate Tri-Amino base solution to powdered form:

Place the Tri-Amino base solution in Pyrex evaporating dish and then into vacuum oven. Set temperature to no more than 95 degrees Centigrade with a vacuum of between one and one and a half negative atmospheres. Evaporate until anhydrous. The mass will be crystalline. Remove from oven, cool and grind to a fine powder (4 microns or finer). The finished product will be white.

### Inhalation Product

To prepare a mixture for therapeutic inhalation via small volume nebulizer, use a 50% dilution of Tri-Amino base liquid to 50% de-ionized water. For an OTC (over the counter) inhalant, to each liter of 50/50 dilution, add 5 Grams of Caffeine & 25 Grams Xylitol.

### Mechanism of Action

The new product, 2-amino 5-triaminoguanidovalericbenzoicdiacetate, releases free amino ions when in an aqua's solution because of weak hydrogen bonding. These ions combine with the nucleic acids within dysfunctional cellular nuclei to achieve the most stable form. The process of biosynthesis causes those unstable nuclei to achieve stability by chemical realignment. The resulting nuclear stabilization causes the reoccurrence of the differentiation process which in turn causes the dysfunctional cells to return to their original function because of already implanted DNA coding. This phenomenon has many positive physiological effects on dysfunctional human and animal cells.

### Experimental Results

Described below are some anecdotal results of some preliminary experiments utilizing Tri-Amino:

### Vaso-Dilation

Tri-Amino has exhibited vaso-dilating qualities and may be used topically or internally to accelerate microcirculation without toxic side effects. This acceleration of microcirculation has many positive benefits.

### Wound Healing

Coupled with the restoration of cellular function, Tri-Amino has exhibited the ability to aid and accelerate the healing of wounds, burns (of all degrees) and several types of chronic lesions. Amongst those lesions healed were radiation burns (third degree with complete suppression of scar formation), squamous cell carcinoma, basal cell carcinoma, psoriasis, venous stasis ulcers, decubitus ulcers (bed sores), deep surgical wounding (with suppression of scar formation), oral & genital herpes

### Hypertension

Encapsulated Tri-Amino has shown the ability to reduce hypertension without toxic side effects.

### Hair Restoration

Tri-Amino lotion has demonstrated the ability to restore follicular function (hair restoration).

### Anti-Inflammatory

Tri-Amino, in all forms of delivery, has shown anti-inflammatory qualities and can relieve flincelosa brought on during the onset of inflammation.

### Pain Relief

Tri-Amino lotion is not an anesthetic but it has the ability to relieve pain without diminishing sensation which is of great value in addressing and treating burns, wounds and other lesions.

### Bleeding Control

Tri-Amino lotion and powder have the property of controlling bleeding.

### Kidney Function

Tri-Amino capsules have restored partial kidney function to a dialysis patient, allowing that person to regularly generate small quantities of urine.

### Relief from Itching

Tri-Amino lotion permanently relieved and controlled the itching that dialysis causes.

### Shrinking Prostate

Tri-Amino liquid has successfully treated an enlarged and inflamed prostate gland causing it to shrink back to normal size.

### Inhalant

Tri-Amino in its OTC (over the counter) inhalant form has consistently shown improved energy/endurance and mental alertness without the side effects of a CNS stimulant.

### Erectile Dysfunction

Tri-Amino lotion has successfully (topically) treated male erectile dysfunction and female sexual dysfunction.

### Hemorrhoids

Tri-Amino lotion has successfully treated hemorrhoids and may be compounded into suppositories.

### Cosmetics

Tri-Amino is a hygroscopic powder and as such is a valuable additive to moisturizing cosmetics.

### Acne Control

Tri-Amino lotion has successfully treated Acne.

### Veterinary Medicine

Tri-Amino has numerous uses in the area of veterinary medicine ranging from topical disturbances to the infusible treatment of mastitis and udder edema in dairy cattle and goats.

### Safety Studies

Applicant has tested the final reaction product in LD-50 animal (laboratory mice) studies. These studies were conducted with oral feeding, intravenous infusion and inhalation. The results showed no toxicity what-so-ever. (Applicant's testing facility was unable to kill the mice with very high concentrations (up to 50 percent liquid or 100 percent powder) of Applicant's Tri-Amino.

While there have been shown what are presently considered to be preferred embodiments of the present invention, it will be apparent to those skilled in the art that various changes and modifications can be made herein without departing from the scope and spirit of the invention. For example, persons skilled in this art may be able based on the disclosures herein recognize additional techniques for producing the intermediate reaction products and/or the final reaction product described herein. Applications of the reaction products (in additions to the ones suggested herein) will undoubtedly become apparent to persons skilled in the art. Therefore, the scope of the patent should be determined by the appended claims and their legal equivalence and not by the examples that have been given.

What is claimed is:

1. A free amino ion releasing product useful for a wide variety of medical and cosmetic applications comprising a hydrogen bonded molecule defined by the chemical formula C₁₅H₂₈N₆O₆.

2. The amino ion releasing product as in claim 1 wherein the chemical name of the said molecule in acetate form is 2-amino 5-triaminoguanidovalericbenzoicdiacetate.

3. The amino ion releasing product as in claim 1 wherein said molecule has a chemical structure is described in FIG. 5.

4. A method of preparing a free amino ion releasing product useful for a wide variety of medical and cosmetic applications comprising a hydrogen bonded molecule, said method comprising the steps of:
A) mixing arginine with water then adding glycine to produce an intermediate product, 2-amino 5-diaminoguanidovalericdiacetate; and
B) mixing the 2-amino 5-diaminoguanidovalericdiacetate with p-aminobenzoic acid (PABA) to yield the final product of the present invention, namely 2-amino 5-triaminoguanidovalericbenzoicdiacetate.

5. A method of preparing a free amino ion releasing product useful for a wide variety of medical and cosmetic applications comprising a hydrogen bonded molecule, said method comprising the steps of:
A) mixing glycine with water under specific conditions,
B) combining the resulting product with p-aminobenzoic acid (PABA) to produce an intermediate product, diaminobenzoic acetate; and
C) combining the diaminobenzoic acetate with arginine to yield 2-amino 5-triaminoguanidovalericbenzoicdiacetate.

6. A method of treatment of a human or animal patient comprising the administration of Tri Amino to said patient.

7. The method as in claim 6 wherein said Tri-Amino is administered orally.

8. The method as in claim 6 wherein said Tri-Amino is administered topically.

9. The method as in claim 6 wherein said Tri-Amino is administered intravenously.

10. The method as in claim 6 wherein said Tri-Amino is administered intra-arterially.

11. The method as in claim 6 wherein said Tri-Amino is inhaled.
